(19) United States
(12) Patent Application Publication
(10) Pub. No.: US 2012/0179225 A1
(43) Pub. Date: Jul. 12, 2012

(54) Vaginal Stimulator Device and Use Thereof for the Treatment of Female Urinary Incontinence

(75) Inventor: Gérard Guez, Paris (FR)

(73) Assignee: ANTEMIS, Celles-Sur-Durolle (FR)

(21) Appl. No.: 13/387,304

(22) PCT Filed: Jul. 29, 2010
(86) PCT No.: PCT/IB2010/001881

§ 371 Date: Apr. 3, 2012

Related U.S. Application Data
(60) Provisional application No. 61/229,886, filed on Jul. 30, 2009.

(51) Int. Cl. A61N 1/36

(52) U.S. Cl. 607/59; 607/72

(57) ABSTRACT

The invention relates to a vaginal stimulator device for the treatment of female urinary incontinence. The device which comprises a stimulator provided with a microcontroller and an electronic circuit for receiving instructions for treating incontinence, electrodes for transmitting electrical pulses to the pelvic floor musculature and instructions to the microcontroller, and battery, wherein said stimulator is integral with a grip member accommodating an antenna which cooperates with a remote control.

This application claims benefit of U.S. 61/229,886, filed Jul. 30, 2009, the entire contents of which is incorporated herein by reference.

The invention relates to a vaginal stimulator device for treating female urinary incontinence.

It is known that the continence organs are controlled by the pelvic floor muscles. These muscles support the viscera and are involved in sphincter function.

Changes in how they function result in problems which considerably affect the quality of life of the individuals concerned and represent significant expense for public health systems.

The interest in having means for maintaining continence and ensuring the well-being of the patients concerned can thus be understood.

Investigations in this field by the inventor have led him to develop a device capable of electrically stimulating the pelvic floor musculature in order to treat stress incontinence and/or have a nervous reflex action providing rapid, effective treatment of urge incontinence.

The invention relates to a vaginal stimulator device comprising a stimulator appropriately shaped to be inserted in the vagina and provided with a microcontroller and an electronic circuit capable of receiving instructions for treating stress incontinence, urge incontinence, or both; electrodes for transmitting the instructions to the microcontroller and for transmitting electrical pulses to the pelvic floor musculature; a battery, advantageously a rechargeable battery.

According to the invention, the device is characterised in that the stimulator is integral with a grip member accommodating an antenna which co-operates with a remote control in such a way as, more particularly, to set the programme for a session depending on a given incontinence treatment and on the duration of the session, and/or to collect feedback information.

The antenna accommodated in the grip member receives instructions from the operator via the remote control and thus sets, in particular, the starting or stopping of the device, the frequency and/or the working or duty cycle and/or the amperage between a null and a maximum value.

If appropriate, the antenna can also transmit feedback information to a recorder via the remote control.

The device is designed in such a way that, when the stimulator is positioned in the vagina, the grip member remains outside the body. Inserting and removing the stimulator are thus facilitated. In addition, since the antenna remains outside, the waves do not penetrate the patient's body.

Advantageously, the grip member of the stimulator is also configured so that the stimulator can be inserted in such a way as to ensure contact of the electrodes with the vaginal mucosa.

Accordingly, it is possible to insert and position the stimulator in a given direction so as to achieve maximum contact with the mucosa and optimum safety for the physiological tissues.

According to an advantageous embodiment of the invention, the device further comprises a case including a compartment in which the stimulator can be secured in position, protected and transported without being damaged and, if appropriate, a compartment for the remote control.

The case also comprises contacts arranged to be in contact with the electrodes of the stimulator, so as to charge the battery accommodated in the stimulator.

The case may contain a recorder for recording the parameters of use, allowing both the practitioner and the patient to monitor the treatment.

The case can be connected to an AC power adapter and is provided with charge circuitry to recharge the battery of the stimulator, when the latter is not in use, via the contacts of the case and the electrodes of the stimulator.

Other features and advantages of the invention will become apparent from the following description of a particular embodiment thereof, which relates to a vaginal stimulator with a grip member.

According to the invention, the device comprises a vaginal stimulator with a grip member, and a case including a compartment for the stimulator.

The stimulator, which is about 8 cm long, is cylindrical in shape and rounded at the end remote from the grip member. This shape is particularly suited to the anatomy of the patient's vagina and makes the stimulator more easily acceptable to her.

The stimulator has two rings on its surface. The grip member arranged at one of its ends assists with the insertion or removal of the stimulator, and the grip member accommodates an antenna which co-operates with a remote control.

The case comprises a base and a flap cover.

The base, made from a rigid material, includes a compartment for the stimulator with the grip member and, if appropriate, for the remote control and two opposite lateral flanges.

Optionally, a pressure part arranged on the inside of the flap cover is so positioned on the flap that, when the case is closed, it presses on the stimulator. This pressure part thus helps maintain the stimulator in the right position and also ensures contact between the electrodes of the stimulator and the contacts arranged in a compartment of the base. The case is closed by folding the cover down onto the base.

The components of the device contained in the case may then be transported without risk of damage.

Accordingly, the invention provides an effective vaginal stimulator device which is practical to use. After several sessions, as prescribed by the practitioner, the patient rapidly notices an improvement in her condition with a reduction in the loss of urine until there is absolutely none at all.

The use of the present device for the treatment of female urinary incontinence also falls within the scope of the invention.

In particular, the invention relates to a method of treating female urinary incontinence characterised by the use of a device as described above, according to appropriate instructions, for a given type of incontinence.

1. A vaginal stimulator device for the treatment of female urinary incontinence, comprising a stimulator appropriately shaped to be inserted in the vagina, said stimulator being provided with a microcontroller and an electronic circuit capable of receiving instructions for treating stress incontinence, urge incontinence or mixed incontinence; electrodes for transmitting electrical pulses to the pelvic floor musculature, said electrodes transmitting the instructions to the microcontroller; a battery, if appropriate a rechargeable battery, wherein the stimulator is integral with a grip member accommodating an antenna which co-operates with a remote control in such a way as to set the programme for a session depending on a given incontinence treatment and to the duration of the session, and/or to collect feedback information.

2. The device according to claim 1, wherein the antenna receives instructions via the remote control and thus sets, in particular, the starting or stopping of the device, the frequency and/or the working or duty cycle and/or the amperage between a null value and a maximum value.

3. The device according to claim 1, wherein the antenna transmits feedback information to a recorder via the remote control.

4. The device according to claim 1, wherein the device is designed in such a way that the grip member remains outside the body when the device is positioned in the vagina.

5. The device according to claim 1, wherein the grip member of the stimulator is configured so that the stimulator can be inserted in such a way as to ensure contact of the electrodes with the vaginal mucosa.

6. The device according to claim 1, wherein the device further comprises a case including a compartment in which the stimulator can be secured in position, protected and transported without being damaged and, if appropriate, a compartment for the remote control.

7. The device according to claim 6, wherein the case includes a recorder.

8. The device according to claim 6, wherein the case connected to the AC power adapter is provided with charge circuitry.

9. The use of the device according to claim 1, for the treatment of female urinary incontinence.

10. A method for the treatment of female urinary incontinence comprising the steps of
inserting a vaginal stimulator device into the vagina of a female in need thereof, said stimulator being provided with a microcontroller and an electronic circuit capable of receiving instructions for treating stress incontinence, urge incontinence or mixed incontinence; electrodes for transmitting electrical pulses to the pelvic floor musculature, said electrodes transmitting the instructions to the microcontroller; a battery, if appropriate a rechargeable battery, wherein the stimulator is integral with a grip member accommodating an antenna which co-operates with a remote control in such a way as to set the programme for a session depending on a given incontinence treatment and to the duration of the session, and/or to collect feedback information; and
transmitting electrical pulses from the device into a pelvic floor musculature of the female.

11. The method of claim 10, further comprising the step of sending instructions to that antenna via the remote control such that starting or stopping of the device, the frequency and/or the working or duty cycle and/or the amperage between a null value and a maximum value is remotely controllable.

12. The method of claim 10, wherein the step of inserting a vaginal stimulator device into the vagina further comprises positioning the device such the the grip member remains outside the body when the device is positioned in the vagina.

13. The method of claim 10, wherein the step of inserting a vaginal stimulator device into the vagina further comprises positioning the grip member such that the stimulator is inserted in such a way as to ensure contact of the electrodes with the vaginal mucosa.

14. The method of claim 10, further comprising the step of transmitting feedback from the antenna to a recorder via the remote control.
